(19) United States
(12) Patent Application Publication
(10) Pub. No.: US 2020/0062684 A1
(43) Pub. Date: Feb. 27, 2020

(54) Anti-Inflammatory Composition

(71) Applicants: House Wellness Foods Corporation, Hyogo (JP); House Foods Group Inc., Osaka (JP)

(72) Inventors: Kengo Kawasaki, Itami-shi, Hyogo (JP); Chinatsu Hanafusa, Itami-shi, Hyogo (JP); Morihiro Aoyagi, Higashiosaka-shi, Osaka (JP); Koichi Taoka, Itami-shi, Hyogo (JP)

(73) Assignees: House Wellness Foods Corporation, Hyogo (JP); House Foods Group Inc., Osaka (JP)

(21) Appl. No.: 16/612,628

(22) PCT Filed: May 11, 2018
(86) PCT No.: PCT/JP2018/018293

§ 371 Date: Nov. 11, 2019

(30) Foreign Application Priority Data
May 12, 2017 (JP) 2017-095713

(51) Int. Cl. C07C 49/248; C07C 49/242

(52) U.S. Cl. CPC C07C 49/248; C07C 49/242

(57) ABSTRACT

It is an object of the present invention to provide a composition having an anti-inflammatory action.

The anti-inflammatory composition of the present invention comprises, as an active ingredient, a compound represented by the following Formula 1. In the following Formula 1, R₁ represents hydrogen or a hydroxy group, and A represents a phenyl group optionally substituted with a hydroxyl group or a methyl group, or a cyclohexenyl group substituted with a hydroxyl group and a methylene group.

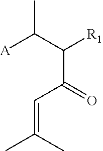

### TECHNICAL FIELD

The present invention relates to an anti-inflammatory composition, a prostaglandin E2 production inhibitory composition, and a nitric oxide production inhibitory composition, which are useful as food or beverage or pharmaceutical products.

The present invention also relates to a novel compound having a prostaglandin E2 production inhibitory activity and a nitric oxide production inhibitory activity.

### BACKGROUND ART

Prostaglandin E2 (PGE2) is generated in leukocytes (macrophages), mast cells, endothelial cells, platelets and the like. Arachidonic acid existing as a structural component of a cell membrane phospholipid is cleaved by phospholipase, and is passed through a cyclooxygenase pathway, thereby synthesizing PGE2. In the process of inflammation, the aforementioned pathway is activated, and generation of PGE2 is thereby increased.

PGE2 released from specific cells acts on target cells present in the vicinity thereof, and induces an inflammatory reaction in the target cells. PGE2 is one chemical mediator for amplifying an inflammatory reaction in an inflamed site, and activates the inflammatory reaction in the inflamed site.

Nitric oxide (NO) is generated in the process of inflammation by type II nitric oxide synthase (iNOS) of leukocytes (macrophages) induced by an inflammatory cytokine or bacterial endotoxin (Non Patent Literature 1). Excessively generated NO is converted to peroxynitrous acid, which then exhibits a cytotoxic action such as DNA damage or LDL oxidation (Non Patent Literature 2). Thus, it is important to suppress excessive generation of NO due to inflammation. In addition, since NO activates an intracellular signal pathway for promoting inflammation, such as an NF-κB pathway (Non Patent Literature 3), suppression of NO generation is also important for exhibiting an anti-inflammatory action (Patent Literature 1).

Inflammation is provoked by stimulating factors such as infections, external wounds or foreign objects Inflammation is a defense reaction of eliminating the stimulating factors and own cells and/or tissues that have become necrotic by such stimulating factors. The inflammatory reaction assists the removal of harmful stimulations such as an infection. On the other hand, inflammation may damage even normal tissues, it may damage living bodies. Hence, it is necessary to suppress excessive inflammatory reactions.

A PGE2 production inhibitor and an anti-inflammatory agent, each comprising a natural compound as an active ingredient, are, for example, described in Patent Literature 2. Moreover, Patent Literature 1 discloses that a mixture of soybeans or an extract thereof and chlorophyll, which has been activated by a light irradiation treatment and/or a heat treatment, has an activity of suppressing NO generation and is useful as an anti-inflammatory agent.

On the other hand, Curcuma longa comprises a large number of sesquiterpene compounds. As such Curcuma longa-derived sesquiterpene compounds, a large number of bisabolane compounds such as Turmeronol A and Turmeronol B have been known (Non Patent Literature 4).

### CITATION LIST

### Patent Literature

Patent Literature 1: International Publication WO 2012/177969
Patent Literature 2: JP Patent Publication (Kokai) No. 2012-056952 A

### Non Patent Literature

Non Patent Literature 1: Robbins basic of Pathology (8th edition), Chapter 2, pp. 58-59
Non Patent Literature 2: J Physiol Pharmacol. 2003; 54(4): 469-87.
Non Patent Literature 3: Curr Drug Targets Inflamm Allergy. 2005; 4(4): 471-9.
Non Patent Literature 4: S. Li et al., Pharmaceutical Crops, 2011, 2, 28-54

### SUMMARY OF INVENTION

### Technical Problem

It is an object of the present invention to provide an anti-inflammatory composition, a PGE2 production inhibitory composition, or a NO production inhibitory composition.

### Solution to Problem

The present invention includes the following inventions.

(1) An anti-inflammatory composition comprising, as an active ingredient, a compound represented by the following Formula 1:

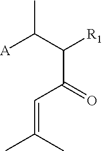

or a salt thereof,
wherein
R₁ represents hydrogen or a hydroxy group, and
A represents a group represented by the following Formula 2:

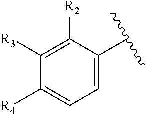

or

### Formula 3:

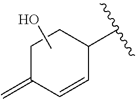

wherein
R₂ represents hydrogen or a hydroxy group,
R₃ represents hydrogen or a hydroxy group,
R₄ represents methyl, hydrogen or a hydroxy group, and
at least one of R₂, R₃ and R₄ represents a hydroxy group.
(2) A prostaglandin E2 production inhibitory composition comprising, as an active ingredient, the compound represented by the above Formula 1 or a salt thereof.
(3) A nitric oxide production inhibitory composition comprising, as an active ingredient, the compound represented by the above Formula 1 or a salt thereof.
(4) A compound represented by the following Formula 4:

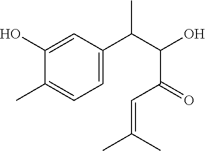 [SMILES: CC(C)=CC(=O)C(O)C(C)c1ccc(C)c(O)c1]

or a salt thereof.
(5) A method for treating or preventing inflammation, comprising administering the compound represented by the above Formula 1 or a salt thereof to a subject such as a human.
(6) The compound represented by the above Formula 1 or a salt thereof, for use in treating or preventing inflammation in a subject such as a human.
(7) Use of the compound represented by the above Formula 1 or a salt thereof for the production of a pharmaceutical composition for treating or preventing inflammation in a subject such as a human.
(8) Non-medical use of the compound represented by the above Formula 1 or a salt thereof in a food or beverage composition for treating or preventing inflammation.
(9) A method for suppressing the production of prostaglandin E2, comprising administering the compound represented by the above Formula 1 or a salt thereof to a subject such as a human.
(10) The compound represented by the above Formula 1 or a salt thereof, for use in suppressing the production of prostaglandin E2 in a subject such as a human.
(11) Use of the compound represented by the above Formula 1 or a salt thereof for the production of a pharmaceutical composition for suppressing the production of prostaglandin E2 in a subject such as a human.
(12) Non-medical use of the compound represented by the above Formula 1 or a salt thereof in a food or beverage composition for suppressing the production of prostaglandin E2.
(13) A method for treating or preventing a disease that is ameliorated or prevented by suppressing the production of prostaglandin E2, the method comprising administering the compound represented by the above Formula 1 or a salt thereof to a subject such as a human.
(14) The compound represented by the above Formula 1 or a salt thereof, for use in treating or preventing a disease that is ameliorated or prevented by suppressing the production of prostaglandin E2 in a subject such as a human
(15) Use of the compound represented by the above Formula 1 or a salt thereof for the production of a pharmaceutical composition for treating or preventing a disease that is ameliorated or prevented by suppressing the production of prostaglandin E2 in a subject such as a human.
(16) Non-medical use of the compound represented by the above Formula 1 or a salt thereof in a food or beverage composition for treating or preventing a disease that is ameliorated or prevented by suppressing the production of prostaglandin E2.
(17) A method for suppressing the production of nitric oxide, comprising administering the compound represented by the above Formula 1 or a salt thereof to a subject such as a human.
(18) The compound represented by the above Formula 1 or a salt thereof, for use in suppressing the production of nitric oxide in a subject such as a human.
(19) Use of the compound represented by the above Formula 1 or a salt thereof for the production of a pharmaceutical composition for suppressing the production of nitric oxide in a subject such as a human.
(20) Non-medical use of the compound represented by the above Formula 1 or a salt thereof in a food or beverage composition for suppressing the production of nitric oxide.
(21) A method for treating or preventing a disease that is ameliorated or prevented by suppressing the production of nitric oxide, the method comprising administering the compound represented by the above Formula 1 or a salt thereof to a subject such as a human.
(22) The compound represented by the above Formula 1 or a salt thereof, for use in treating or preventing a disease that is ameliorated or prevented by suppressing the production of nitric oxide in a subject such as a human.
(23) Use of the compound represented by the above Formula 1 or a salt thereof for the production of a pharmaceutical composition for treating or preventing a disease that is ameliorated or prevented by suppressing the production of nitric oxide in a subject such as a human.
(24) Non-medical use of the compound represented by the above Formula 1 or a salt thereof in a food or beverage composition for treating or preventing a disease that is ameliorated or prevented by suppressing the production of nitric oxide.
(25) A food or beverage composition comprising the compound represented by the above Formula 4 or a salt thereof and other components acceptable as food or beverage. The content of the compound represented by the above Formula 4 or a salt thereof is preferably an effective amount, in which when the above-described food or beverage composition is orally administered to a human, it generates one or more actions selected from an anti-inflammatory action, a prostaglandin E2 production inhibitory action, and a nitric oxide inhibitory action, in the body of the human; and is more preferably 0.0001% by weight or more, 0.001% by weight or more, 0.01% by weight or more, 0.1% by weight or more, or 1% by weight or more, with respect to the total amount of the above-described food or beverage composition.
(26) A pharmaceutical composition comprising the compound represented by the above Formula 4 or a salt thereof and other components acceptable as pharmaceutical products. The content of the compound represented by the above Formula 4 or a salt thereof is preferably an effective amount, in which when the above-described pharmaceutical composition is administered to a subject such as a human, it generates one or more actions selected from an anti-inflammatory action, a prostaglandin E2 production inhibitory action, and a nitric oxide inhibitory action, in the body of the subject; and is more preferably 0.0001% by weight or more, 0.001% by weight or more, 0.01% by weight or more, 0.1% by weight or more, or 1% by weight or more, with respect to the total amount of the above-described pharmaceutical composition.

The present application claims priority from Japanese Patent Application No. 2017-095713; the disclosure of which is hereby incorporated by reference.

### Advantageous Effects of Invention

The composition of the present invention is useful as an anti-inflammatory agent, a PGE2 production inhibitor, or a NO production inhibitor.

The compound of the present invention has an anti-inflammatory activity, a PGE2 generation inhibitory activity, or an NO generation inhibitory activity.

### BRIEF DESCRIPTION OF DRAWINGS

FIG. 1 shows the PGE2 concentration in the culture supernatant of RAW264.7 treated with Turmeronol A.

FIG. 2 shows the PGE2 concentration in the culture supernatant of RAW264.7 treated with 2-methyl-6-(4-hydroxyphenyl)-2-hepten-4-one.

FIG. 3 shows the PGE2 concentration in the culture supernatant of RAW264.7 treated with 4-methylene-5-hydroxybisabola-2,10-dien-9-one.

FIG. 4 shows the PGE2 concentration in the culture supernatant of RAW264.7 treated with the component D-b.

FIG. 5 shows the PGE2 concentration in the culture supernatant of RAW264.7 treated with Turmeronol B.

FIG. 6 shows the NO₂⁻ concentration in the culture supernatant of RAW264.7 treated with Turmeronol A.

FIG. 7 shows the NO₂⁻ concentration in the culture supernatant of RAW264.7 treated with 2-methyl-6-(4-hydroxyphenyl)-2-hepten-4-one.

FIG. 8 shows the NO₂⁻ concentration in the culture supernatant of RAW264.7 treated with 4-methylene-5-hydroxybisabola-2,10-dien-9-one.

FIG. 9 shows the NO₂⁻ concentration in the culture supernatant of RAW264.7 treated with the component D-b.

FIG. 10 shows the NO₂⁻ concentration in the culture supernatant of RAW264.7 treated with Turmeronol B.

### DESCRIPTION OF EMBODIMENTS

### <Active Compound>

The composition of the present invention comprises the compound represented by the above Formula 1 or a salt thereof as an active ingredient having an anti-inflammatory activity, a PGE2 generation inhibitory activity, and an NO generation inhibitory activity. In the following explanation, the compound represented by Formula 1 or a salt thereof may also be referred to as an “active compound”.

The compound represented by Formula 1 may be a compound having a planar structure represented by Formula 1. The configuration is not particularly limited, and it may also be a mixture of compounds having several types of configurations. It is to be noted that, in Formula 2 and Formula 3, the bond interrupted with a wavy line indicates the binding of A to carbon in Formula 1.

A more preferred embodiment of the compound of Formula 1, wherein A is the group represented by Formula 2, will be described.

In Formula 2, R₄ is preferably a methyl or hydroxy group. In a preferable embodiment of Formula 2, only one of R₂, R₃ and R₄ is a hydroxy group. In a more preferable embodiment of Formula 2, only one of R₂, R₃ and R₄ is a hydroxy group and R₄ is a methyl or hydroxy group.

The compound of Formula 1, wherein A is the group represented by Formula 2, is more preferably a compound having any one of the following planar structures.

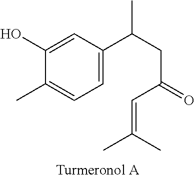 [SMILES: CC(C)=CC(=O)CC(C)c1ccc(C)c(O)c1]

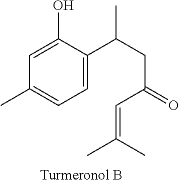 [SMILES: CC(C)=CC(=O)CC(C)c1ccc(C)cc1O]

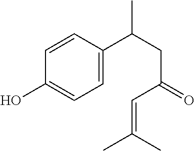 [SMILES: CC(C)=CC(=O)CC(C)c1ccc(O)cc1]

2-Methyl-6-(4-hydroxyphenyl)-2-heptene-4-one

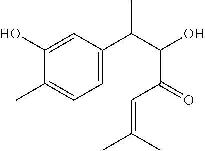 [SMILES: CC(C)=CC(=O)C(O)C(C)c1ccc(C)c(O)c1]

Component D-b(2-methyl-5-hydroxy-6-(3-hydroxy-4-methylphenyl)-2-hepten-4-one)

Among these, the component D-b is a novel compound, which the present inventors have separated from a Curcuma longa extract and then have identified. The component D-b can be nominated as 2-methyl-5-hydroxy-6-(3-hydroxy-4-methylphenyl)-2-hepten-4-one.

In natural products of Turmeronol A, Turmeronol B, and 2-methyl-6-(4-hydroxyphenyl)-2-hepten-4-one separated from the Curcuma longa extract, the carbon at position 6 in the partial structure of 2-methyl-2-hepten-4-one has been known to be in S-configuration. However, in the above-described more preferred example of the compound of Formula 1, it is adequate if the compound may have the above-described planar structure, and the steric structure thereof is not particularly limited.

A more preferred embodiment of the compound of Formula 1, wherein A is the group represented by Formula 3, will be described.

The group represented by Formula 3 is more preferably a group represented by the following Formula 3-1:

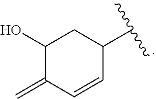 [SMILES: *C1C=CC(=C)C(O)C1]

The group represented by Formula 3 or Formula 3-1 may be a group having a planar structure represented by Formula 3 or Formula 3-1. The configuration thereof is not particularly limited, and it may comprise groups having several types of configurations.

The compound of Formula 1, wherein A is the group represented by Formula 3, is more preferably a compound having the following planar structure:

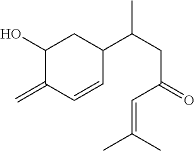 [SMILES: C=C1C=CC(C(C)CC(=O)C=C(C)C)CC1O]

4-Mehylene-5-hydroxybisabola-2,10-diene-9-one

The salt of the compound represented by Formula 1 is not particularly limited, as long as it is a pharmaceutically acceptable salt. An example of the pharmaceutically acceptable salt may be a sodium salt (a sodium salt of a phenolic hydroxyl group).

### <Method for Producing Active Compound>

The active compound used in the present invention may be either a plant-derived active compound, or an artificially synthesized active compound. For example, an optically active (+)-Turmeronol A can be synthesized according to the method described in Biosci Biotechnol Biochem. 1993; 57(7): 1137-40.

The active compound used in the present invention is more preferably derived from a plant material, and is further preferably derived from a Zingiberaceae Curcuma plant. Examples of such a Zingiberaceae Curcuma plant may include Curcuma longa, Curcuma aromatica, Curcuma zedoaria, Curcuma phaeocaulis, Curcuma kwangsiensis, Curcuma wenyujin, and Curcuma xanthorrhiza. Among these, Curcuma longa is preferable. The active compound can be obtained from the rhizome or other parts of a Zingiberaceae Curcuma plant. As such a rhizome, a rhizome collected from the soil may be used, or a suitable part of the rhizome may be directly used. Such a collected rhizome may be cut into an appropriate size or shape, or it may be converted to the form of a disintegrated product and may be then used. A plant material may be dried, as appropriate.

The active compound can be extracted from a plant material comprising the same. As an extraction solvent, a polar organic solvent (methanol, ethanol, etc.), water, a non-polar organic solvent (ethyl acetate, etc.) can be used. In particular, the plant extract comprising the active compound is preferably a water extract obtained from a plant material according to water extraction or a methanol/water extract obtained by further extracting the water extract with a methanol/water mixed solvent, and more preferably the methanol/water extract. As such water, hot water at 95° C. or higher is preferably used. The plant extract is used after the extraction solvent has been volatilized and removed, as necessary. The plant extract comprising an active compound may also be directly mixed into the composition of the present invention.

Furthermore, an active compound fraction that has been highly purified from a plant extract comprising the active compound may be mixed into the composition of the present invention. For example, a plant extract comprising the active compound may be subjected to liquid-liquid distribution with ethyl acetate/water, so that the active compound can be highly purified in the ethyl acetate fraction. Furthermore, a plant extract comprising the active compound or a fraction thereof may be subjected to a purification treatment involving chromatography, so that a highly purified active compound can be obtained. Such chromatography may, for example, be reverse phase column chromatography, or normal phase thin-layer chromatography.

A processing such as drying, powdering, granulation, or fluidization may be performed on a plant extract comprising the active compound or a fraction thereof according to a common method.

The active compound is preferably purified.

### <Composition of the Present Invention and Intended Use Thereof>

The composition of the present invention may be either the above-described active compound itself, or a composition comprising the active compound and at least one further component. The composition of the present invention comprising the active compound and the at least one further component may be prepared by mixing the active compound with the at least one further component. The composition may also be prepared by formulating the active compound and the at least one further component according to suitable means. The composition may also be prepared by formulating the active compound and the at least one further component, which is then further mixed with additional components. Herein, the active compound may be in the form of the above-described plant extract comprising the active compound, or a fraction thereof. In the present invention, the form of the composition comprising the active compound is not particularly limited, and for example, the composition may be a liquid, a fluid, a gel, a semi-solid or a solid composition.

The above-described at least one further component is not particularly limited. It is preferably a component that is acceptable in a final product such as a food or beverage product or a pharmaceutical product, and is more preferably an orally ingestible component.

Examples of such further component may include sweeteners, acidulants, vitamins, minerals, thickeners, emulsifiers, antioxidants, and water. Moreover, as necessary, additional components such as pigments, perfumes, preservatives, antiseptics, fungicides, or further physiologically active substances may be added.

Examples of the sweeteners may include: monosaccharides or disaccharides, such as glucose, fructose, sucrose, lactose, maltose, palatinose, trehalose, or xylose; high-fructose corn syrup (glucose fructose liquid sugar, fructose glucose liquid sugar, sugar-mixed corn syrup, etc.); sugar alcohol (erythritol, xylitol, lactitol, palatinit, sorbitol, reduced starch syrup, etc.); honey; and high-intensity sweeteners (sucralose, acesulfame potassium, thaumatin, stevia, aspartame, etc.).

Examples of the acidulants may include citric acid, malic acid, gluconic acid, tartaric acid, lactic acid, phosphoric acid, and the salts thereof. These acidulants can be used alone or in combination of two or more types.

Examples of the vitamins may include vitamin A, vitamin B 1, vitamin B2, vitamin B6, vitamin E, niacin, and inositol.

Examples of the minerals may include calcium, magnesium, zinc, and iron.

Examples of the thickeners may include carrageenan, gellan gum, xanthan gum, gum Arabic, tamarind gum, guar gum, locust bean gum, karaya gum, agar, gelatin, pectin, soybean polysaccharides, and carboxymethyl cellulose (CMC).

Examples of the emulsifiers may include glycerin fatty acid ester, sucrose fatty acid ester, sorbitan fatty acid ester, lecithin, vegetable sterol, and saponin.

Examples of the antioxidants may include vitamin C, tocopherol (vitamin E), enzymatically modified rutin, and catechin.

The above-described further components can be each mixed into a composition, such as a food or beverage product or a pharmaceutical product, as appropriate, in a generally adopted range, by a person skilled in the art.

The composition formulated from the active compound and at least one further component by suitable means may be a solid composition, such as powders, granules, a capsule, or a tablet (a coated tablet such as a sugar-coated tablet, a multilayered tablet, an oral disintegrant, a chewable tablet, etc.), or may also be a liquid composition such as a solution agent.

The composition of the present invention is preferably a food or beverage product or a pharmaceutical product, and is more preferably a food or beverage product. The term “food or beverage product” used herein may include a food additive and a food or beverage raw material. The food or beverage raw material is used for production of a food or beverage by combining it with additional food materials. When the composition comprising the active compound is a food or beverage product or a food or beverage raw material, the “food or beverage” may preferably be a food with functional claims, a food for specified health uses, or a supplement for nutrition supply.

The compound represented by the above Formula 1 or a salt thereof is administered to a subject such as a human, so that inflammation can be treated or prevented in the subject. Herein, the compound represented by the above Formula 1 or a salt thereof is administered in an effective amount for treating or preventing inflammation. The administration route is preferably oral or transnasal administration, and is particularly preferably oral administration. Hence, the composition of the present invention comprising the compound represented by the above Formula 1 or a salt thereof is useful as an anti-inflammatory composition. The anti-inflammatory composition may be either a pharmaceutical composition, or may also be a composition for non-medical use, such as a food or beverage composition.

The compound represented by the above Formula 1 or a salt thereof is administered to a subject such as a human, so that the production of prostaglandin E2 can be suppressed in the subject. Herein, the compound represented by the above Formula 1 or a salt thereof is administered in an effective amount for suppressing the production of prostaglandin E2. The administration route is preferably oral or transnasal administration, and is particularly preferably oral administration. In the subject to which the compound represented by the above Formula 1 or a salt thereof has been administered, the production of prostaglandin E2 is suppressed in cells such as leukocytes (macrophages), mast cells, endothelial cells, or platelets. Hence, the composition of the present invention comprising the compound represented by the above Formula 1 or a salt thereof is useful as a prostaglandin E2 production inhibitory composition. The prostaglandin E2 production inhibitory composition may be either a pharmaceutical composition, or may also be a composition for non-medical use, such as a food or beverage composition.

The compound represented by the above Formula 1 or a salt thereof is administered to a subject such as a human, so that a disease that is ameliorated or prevented by suppressing the production of prostaglandin E2 can be treated or prevented in the subject. Herein, the compound represented by the above Formula 1 or a salt thereof is administered in an effective amount for treating or preventing the aforementioned disease. The administration route is preferably oral or transnasal administration, and is particularly preferably oral administration. In the subject to which the compound represented by the above Formula 1 or a salt thereof has been administered, the production of prostaglandin E2 is suppressed in cells such as leukocytes (macrophages), mast cells, endothelial cells, or platelets, and the disease can be thereby treated or prevented.

The compound represented by the above Formula 1 or a salt thereof is administered to a subject such as a human, so that the production of nitric oxide can be suppressed in the subject. Herein, the compound represented by the above Formula 1 or a salt thereof is administered in an effective amount for suppressing the production of nitric oxide. The administration route is preferably oral or transnasal administration, and is particularly preferably oral administration. In the subject to which the compound represented by the above Formula 1 or a salt thereof has been administered, the production of nitric oxide is suppressed in cells such as leukocytes (macrophages). Hence, the composition of the present invention comprising the compound represented by the above Formula 1 or a salt thereof is useful as a nitric oxide production inhibitory composition. The nitric oxide production inhibitory composition may be either a pharmaceutical composition, or may also be a composition for non-medical use, such as a food or beverage composition.

The compound represented by the above Formula 1 or a salt thereof is administered to a subject such as a human, so that a disease that is ameliorated or prevented by suppressing the production of nitric oxide can be treated or prevented in the subject. Herein, the compound represented by the above Formula 1 or a salt thereof is administered in an effective amount for treating or preventing the aforementioned disease. The administration route is preferably oral or transnasal administration, and is particularly preferably oral administration. In the subject to which the compound represented by the above Formula 1 or a salt thereof has been administered, the production of nitric oxide is suppressed in cells such as leukocytes (macrophages), and the disease can be thereby treated or prevented.

### EXAMPLES

### 1. Preparation Method

A hot water extract was obtained from the rhizome of Curcuma longa according to hot water extraction. Subsequently, a 90% methanol extract was obtained by extracting the hot water extract with 90% methanol (methanol/water=90/10 (v/v)). Subsequently, the 90% methanol extract was subjected to liquid-liquid distribution with ethyl acetate/water to obtain an ethyl acetate fraction. Turmeronol A and 4-methylene-5-hydroxybisabola-2,10-dien-9-one were purified from the ethyl acetate fraction according to reverse phase column chromatography, and then, were each dissolved in dimethyl sulfoxide, which were then used in the subsequent tests. 2-Methyl-6-(4-hydroxyphenyl)-2-hepten-4-one and the component D-b were purified from the aforementioned ethyl acetate fraction according to reverse phase column chromatography and normal phase thin-layer chromatography, and then, were each dissolved in dimethyl sulfoxide, which were then used in the subsequent tests.

Regarding Turmeronol B, a commercially available product was purchased from Nagara Science Co., Ltd., and was then dissolved in dimethyl sulfoxide, which was the used in the subsequent tests.

### 2. Identification of Individual Components

The structures of individual isolated components were identified based on the results of instrumental analyses such as ¹H NMR, ¹³C NMR and LCMS, and known information.

Turmeronol A and Turmeronol B are described in Agric. Biol. Chem., 1990; 54(9): 2367-71.

2-Methyl-6-(4-hydroxyphenyl)-2-hepten-4-one is described in Chem. Pharm. Bull., 2007; 55(6): 940-3.

4-Methylene-5-hydroxybisabola-2,10-dien-9-one is described in J. Asian Nat. Prod. Res., 2009, 11, 569-575.

The component D-b had the following chemical shifts according to ¹H NMR and ¹³C NMR:

¹H NMR (500 MHz, methanol-d₃) δ ppm: 1.16 (d, J=6.87 Hz, 3H), 1.85 (d, J=1.15 Hz, 3H), 2.07 (d, J=1.00 Hz, 3H), 2.11 (s, 3H), 2.95 (m, J=6.87, 5.73 Hz, 1H), 4.10 (d, J=5.73 Hz, 1H), 6.13-6.15 (m, 1H), 6.63 (dd, J=7.45, 1.72 Hz, 1H), 6.66 (d, J=1.72 Hz, 1H), 6.93 (d, J=7.45 Hz, 1H).

¹³C NMR (126 MHz, methanol-d₃) δ ppm: 14.47, 14.50, 19.79, 26.48, 42.86, 47.14, 47.32, 47.49, 47.66, 47.83, 48.00, 48.16, 81.26, 113.89, 118.65, 120.52, 122.27, 130.14, 142.13, 154.95, 157.59, 201.74.

Moreover, the high resolution mass spectrum (HRMS) of the component D-b was obtained according to high resolution LCMS, and the following accurate mass was obtained. HRMS (ESI) Calcd for C₁₅H₂₁O₃: 249.1485 [M+H]⁺, Found: 249.1480 [M+H]⁺.

From these results, the component D-b was specified to be a novel compound that was 2-methyl-5-hydroxy-6-(3-hydroxy-4-methylphenyl)-2-hepten-4-one.

The planar structures of individual compounds are shown below.

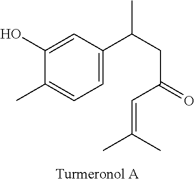 [SMILES: CC(C)=CC(=O)CC(C)c1ccc(C)c(O)c1]

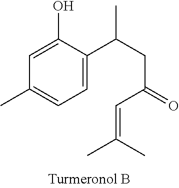 [SMILES: CC(C)=CC(=O)CC(C)c1ccc(C)cc1O]

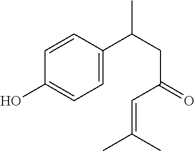 [SMILES: CC(C)=CC(=O)CC(C)c1ccc(O)cc1]

2-Methyl-6-(4-hydroxyphenyl)-2-heptene-4-one

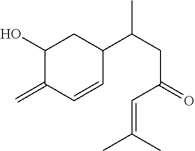 [SMILES: C=C1C=CC(C(C)CC(=O)C=C(C)C)CC1O]

4-Mehylene-5-hydroxybisabola-2,10-diene-9-one

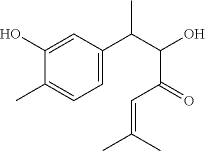 [SMILES: CC(C)=CC(=O)C(O)C(C)c1ccc(C)c(O)c1]

Component D-b (2-methyl-5-hydroxy-6-(3-hydroxy-4-methylphenyl)-2-hepten-4-one)

### 3. Evaluation of Anti-Inflammatory Action

In the following experiments, the mouse macrophage line RAW264.7 was used. The RAW264.7 cells were seeded at a cell density of 1.5×10⁵ cells in a DMEM medium (10% FBS) on a 96-well plate, and were then cultured for 24 hours in a CO₂ incubator, until the cells became confluent. The mouse macrophage line RAW264.7 that had been cultured on the 96-well plate was pre-treated for 1 hour with Turmeronol A, 2-methyl-6-(4-hydroxyphenyl)-2-hepten-4-one, 4-methylene-5-hydroxybisabola-2,10-dien-9-one, the component D-b, or Turmeronol B, in a predetermined concentration (i.e., multiple concentrations selected from 1.7 μg/mL, 3.2 μg/mL, 6.3 μg/mL, 12.5 μg/mL, and 25 μg/mL). Thereafter, 20 ng/mL lipopolysaccharide (LPS, an inflammation-inducing factor) was added to each resulting cells, and the obtained mixture was then cultured for 12 hours. Thereafter, a supernatant was recovered, and the amount of prostaglandin E2 (PGE2) released into the supernatant was measured according to a competitive ELISA method. On the other hand, the amount of NO₂⁻ (an oxidized product of NO, the amount of which reflects the NO amount) released into the supernatant was measured according to a Griess method. When the cells were treated with each component, DMEM that was not supplemented with 10% FBS was used. A test group, in which the same operations as described above were carried out with the exception that the cells were not treated with a Curcuma longa-derived component, was defined as control/LPS(+), whereas a test group, in which the same operations as those of control/LPS(+) were carried out with the exception that LPS was not added into the medium upon the culture for 12 hours, was defined as control/LPS(−).

### 4. Results

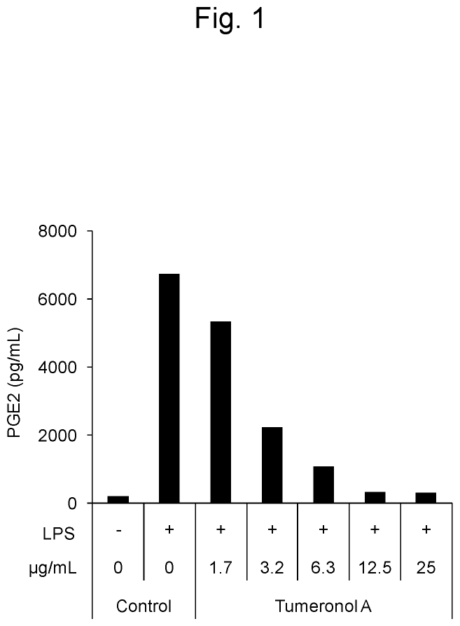

The PGE2 concentration in the culture supernatant of RAW264.7 treated with Turmeronol A is shown in FIG. 1.

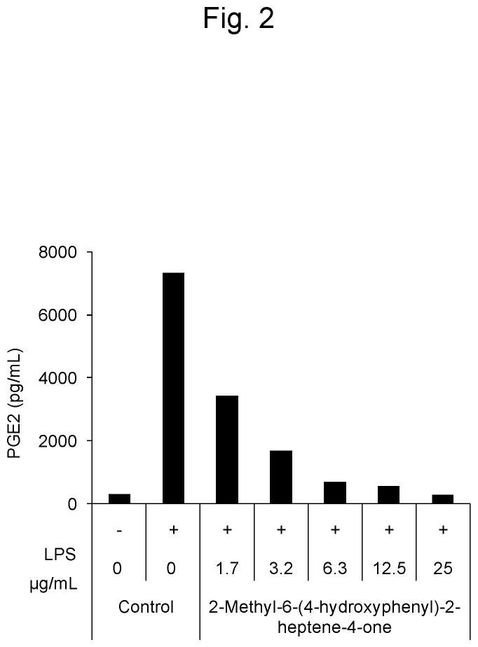

The PGE2 concentration in the culture supernatant of RAW264.7 treated with 2-methyl-6-(4-hydroxyphenyl)-2-hepten-4-one is shown in FIG. 2.

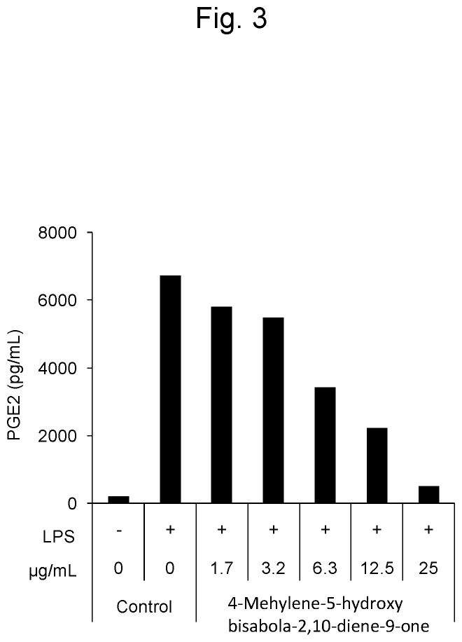

The PGE2 concentration in the culture supernatant of RAW264.7 treated with 4-methylene-5-hydroxybisabola-2,10-dien-9-one is shown in FIG. 3.

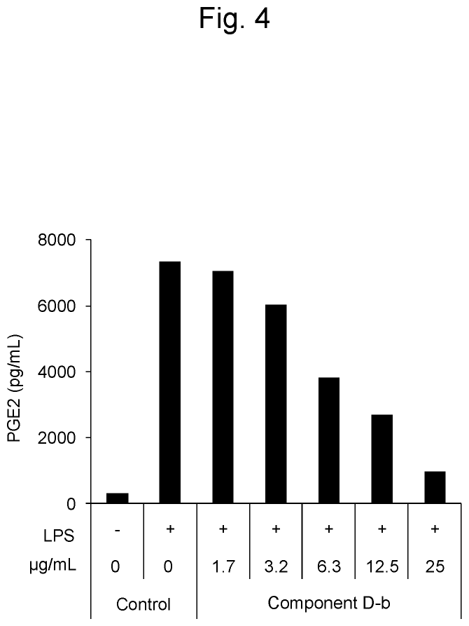

The PGE2 concentration in the culture supernatant of RAW264.7 treated with the component D-b is shown in FIG. 4.

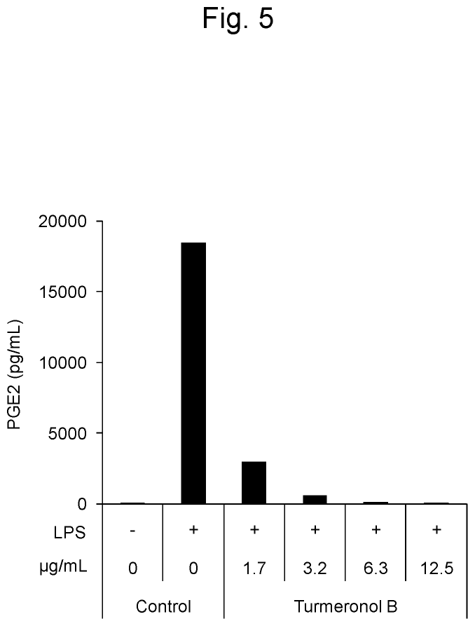

The PGE2 concentration in the culture supernatant of RAW264.7 treated with Turmeronol B is shown in FIG. 5.

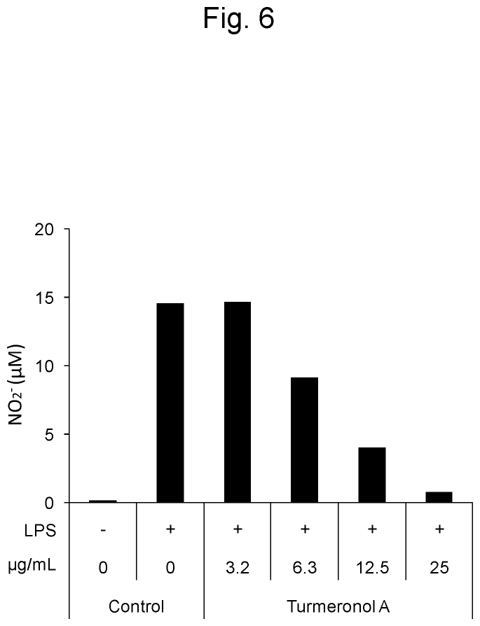

The NO₂⁻ concentration in the culture supernatant of RAW264.7 treated with Turmeronol A is shown in FIG. 6.

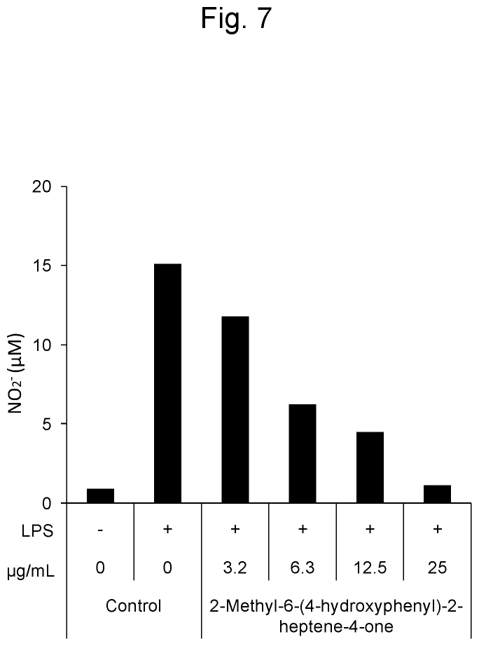

The NO₂⁻ concentration in the culture supernatant of RAW264.7 treated with 2-methyl-6-(4-hydroxyphenyl)-2-hepten-4-one is shown in FIG. 7.

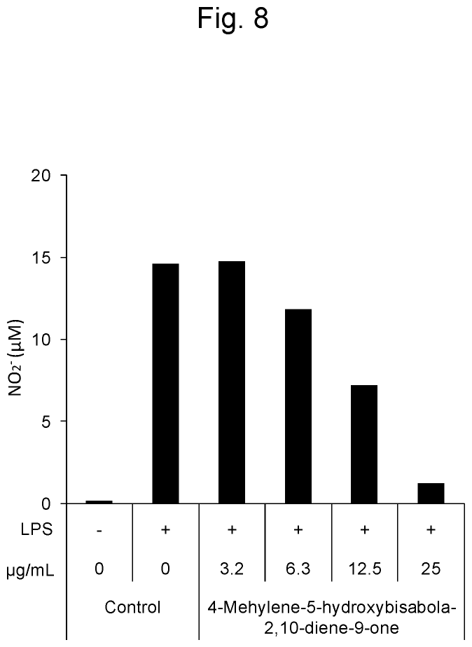

The NO₂⁻ concentration in the culture supernatant of RAW264.7 treated with 4-methylene-5-hydroxybisabola-2,10-dien-9-one is shown in FIG. 8.

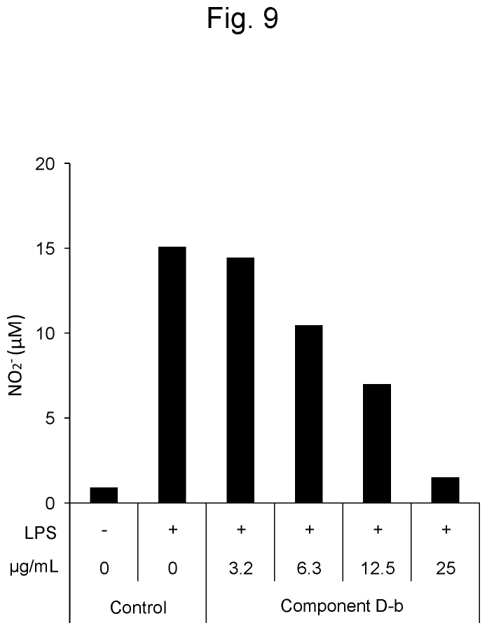

The NO₂⁻ concentration in the culture supernatant of RAW264.7 treated with the component D-b is shown in FIG. 9.

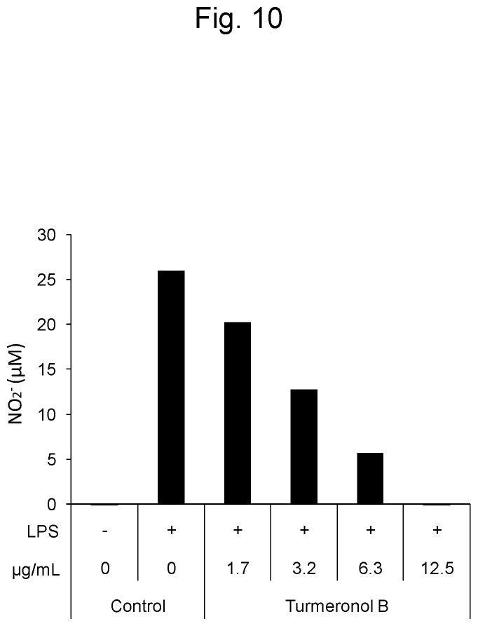

The NO₂⁻ concentration in the culture supernatant of RAW264.7 treated with Turmeronol B is shown in FIG. 10.

In FIGS. 1 to 10, the symbol “+” indicates addition of 20 ng/mL LPS into the medium upon the culture for 12 hours, whereas the symbol “−” indicates non-addition of LPS.

### INDUSTRIAL APPLICABILITY

The composition and compound of the present invention are useful in the field of food or beverage or pharmaceutical products.

All publications, patents and patent applications cited in the present description are incorporated herein by reference in their entirety.

1. An anti-inflammatory composition comprising, as an active ingredient, a compound represented by the following Formula 1: 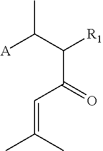
or a salt thereof,
wherein
R₁ represents hydrogen or a hydroxy group, and
A represents a group represented by the following Formula 2: 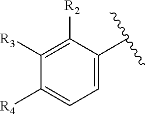
or
Formula 3: 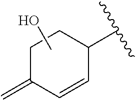
wherein
R₂ represents hydrogen or a hydroxy group,
R₃ represents hydrogen or a hydroxy group,
R₄ represents methyl, hydrogen or a hydroxy group, and
at least one of R₂, R₃ and R₄ represents a hydroxy group.

2. A prostaglandin E2 production inhibitory composition comprising, as an active ingredient, a compound represented by the following Formula 1: 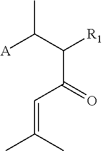
or a salt thereof,
wherein
R₁ represents hydrogen or a hydroxy group, and
A represents a group represented by the following Formula 2: 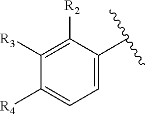
or
Formula 3: 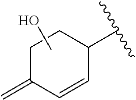
wherein
R₂ represents hydrogen or a hydroxy group,
R₃ represents hydrogen or a hydroxy group,
R₄ represents methyl, hydrogen or a hydroxy group, and
at least one of R₂, R₃ and R₄ represents a hydroxy group.

3. A nitric oxide production inhibitory composition comprising, as an active ingredient, a compound represented by the following Formula 1: 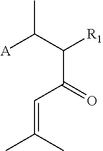
or a salt thereof,
wherein
R₁ represents hydrogen or a hydroxy group, and
A represents a group represented by the following Formula 2: 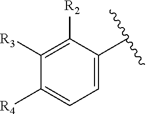
or
Formula 3: 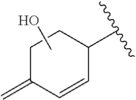
wherein
R₂ represents hydrogen or a hydroxy group,
R₃ represents hydrogen or a hydroxy group,
R₄ represents methyl, hydrogen or a hydroxy group, and
at least one of R₂, R₃ and R₄ represents a hydroxy group.

4. A compound represented by the following Formula 4: 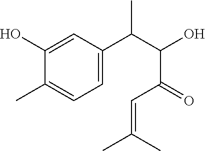 [SMILES: CC(C)=CC(=O)C(O)C(C)c1ccc(C)c(O)c1]
or a salt thereof.
